(19) United States
(12) Patent Application Publication
(10) Pub. No.: US 2012/0226266 A1
(43) Pub. Date: Sep. 6, 2012

(54) LAPAROSCOPIC APPARATUS

(75) Inventors: Ashitava Ghosal, Bangalore (IN); Piyush Goel, Bangalore (IN); Harihar Suresh Kumbhare, Bangalore (IN); Makam Ramesh, Bangalore (IN)

(73) Assignees: B.E.S.T. INSTITUTE AND RESEARCH CENTRE, Bangalore (IN); INDIAN INSTITUTE OF SCIENCE, Bangalore (IN)

(21) Appl. No.: 13/408,733

(22) Filed: Feb. 29, 2012

Related U.S. Application Data
(63) Continuation of application No. PCT/IN2010/000559, filed on Aug. 25, 2010.

(30) Foreign Application Priority Data
Aug. 31, 2009 (IN) 2110/CHE/2009

(51) Int. Cl. A61B 17/00

(52) U.S. Cl. 606/1

(57) ABSTRACT

The present invention provides a laparoscopic apparatus having an elongated barrel connected to a handle case with a fixed handle and a movable handle. A rotatable conduit is connected to a rotatable wheel with a movable rod disposed inside the elongated barrel, which is operable by the rotatable wheel. An end-effector with wrist-like movement is connected to an articulation assembly of the fixed handle in conjunction with a universal joint. The laparoscopic apparatus of the present invention with the end-effector assembly provides a wrist-like lateral movement. The laparoscopic surgical apparatus of the present invention has an improved articulation and an extra degree of freedom, while retaining its existing surgical functions.

### CROSS-REFERENCE TO RELATED PATENT APPLICATIONS

This patent application is a continuation of International Application No PCT/IN2010/000559, filed Aug. 25, 2010, which claims priority to Indian Patent Application No. 2110/CHE/2009, filed Aug. 31, 2009, the entire teachings and disclosure of which are incorporated herein by reference thereto.

### TECHNICAL FIELD

The present invention relates to a surgical device and in particular to a laparoscopic apparatus.

### BACKGROUND OF THE INVENTION

Recent development in the field of robotics has opened a new frontier for innovation and development of surgical tools for use in minimally invasive surgery (MIS). An instrument of this type is used in particular in surgical procedures that use minimally invasive operating techniques. In a minimally invasive operation, a number of small incisions are made, and instruments and the like can be inserted though these incisions to perform operation like grasping, suturing, cutting, tying knots, etc. Minimally invasive surgery technique has the advantages of smaller incision, less pain, faster recovery period, and less exposure of other internal organs.

In addition to the known benefits of the MIS operating technique, a number of drawbacks were also encountered. For example, existing MIS instruments deny the surgeon the flexibility of tool placement found in open surgery. Most of the laparoscopic tool have rigid shafts and are constrained to approach the workspace from the direction of the small incision. Additionally, the length and construction of many laparoscopic instruments reduce the surgeon's ability to feel forces exerted by tissues and organs on laparoscopic instruments. The lack of dexterity and sensitivity provided by laparoscopic tools is a major impediment to the expansion of MIS.

In stapler type of surgical instruments, rotating trigger mechanisms are used to move graspers or jaws of the instruments for their positioning. However, in such instruments no locking devices are present to lock the rotating trigger mechanism at any specified position before actuating the graspers or jaws.

In some surgical tools, there are two working members or fingers, which pivot about a common pivot axis, such as the graspers or forceps. The fingers are actuated to exert a gripping force on tissues or organs. An actuation drive arrangement, such as a pulley and cable system or a push-pull rod, is used to actuate end-effectors. Because the size of the tools is kept small, force is not enough to hold the organs/tissues. For instance, in a pulley and cable system, the force transmission is often increased by increasing the size of the pulley. In the MIS, a significant increase in the size of the pulley used in an end-effector is not desirable.

In trigger controlled laparoscopic surgical tools, bend and rotation of a shaft mimics human wrist-like movement. A “pen click” switch controls opening and closing of the graspers. The mechanism allows simultaneous bending and rotation of an end of the shaft and graspers. However, the bend of the shaft leaves an opening in the tool, making sterilization nearly impossible. This mechanism also has multiple ball bearings in the trigger and the shaft, which cannot be easily sterilized due to the grease needed for smooth movement of the ball bearings.

In a joystick control design, a thumb operated joystick is used for smooth movement of serrated graspers. Movement of the joystick is translated to a ball and socket joint due to simultaneous bend and rotation of a shaft. A trigger mechanism control opens and closes the serrated grasper. However, the problem with this tool is the lack of tactile feedback, which surgeons desire during operation.

### SUMMARY OF THE INVENTION

The present invention provides a laparoscopic surgical apparatus having an elongated barrel connected to a handle case with a fixed handle and a movable handle. A rotatable conduit is provided, which is connected to a rotatable wheel with a movable rod is disposed inside the elongated barrel. The apparatus further comprises a laterally movable end-effector with an operating element on one side and the rotatable conduit connected to the other side. Further, the apparatus comprises a holder with a holder ring connected to the fixed handle and a movable vertical support member with guide actuators movably connected to movable guide members. At least a pair of the movable guide members is axially disposed in an intervening space between the elongated barrel and the rotatable conduit and is connected to the guide actuators and the end-effector. Also, a universal joint is connected to the rotatable conduit on one side and to the end-effector on the other side. Further, a movable and flexible element is connected to the movable rod and the operating element to provide an articulation support to surgical functions of the apparatus. The laparoscopic apparatus, during the course of a laparoscopic surgery, enables the user not only to hold and cut the selected subject but also simultaneously enables the lateral wrist-like movement along with rotary movement of the movable end-effector.

The above summary of the present invention is not intended to describe each illustrated embodiment of the present invention. The other aspects and embodiments of the invention will become apparent upon the review of the following drawings and

### BRIEF DESCRIPTION OF THE DRAWINGS

FIG. 1 is a schematic view of a laparoscopic apparatus showing an end effector with an operating element, according to an in accordance with one embodiment of the subject matter.

FIG. 2 is an exploded view of the laparoscopic apparatus of FIG. 1, showing the arrangement of various parts, according to an embodiment.

FIG. 3 is an exploded view of a fixed handle and some of the parts associated with an actuating mechanism of the laparoscopic apparatus, according to an embodiment.

FIG. 4 is a cross-sectional view of a handle case and the actuating mechanism of FIG. 3 according to an embodiment.

FIG. 5 is a top perspective open view of a rotatable wheel, guide actuators, movable guide members, and a holder ring of the laparoscopic apparatus, according to an embodiment.

FIG. 6 is a top cross-sectional view of the rotatable wheel, the movable guide members, the guide actuators, a rotatable conduit, and an elongated barrel of the laparoscopic apparatus.

FIG. 7 is a view of an end effector assembly of the laparoscopic apparatus, connected to the movable guide members, a universal joint and an operating element, according to an embodiment.

FIGS. 8 and 9 depict a view of a universal joint of the laparoscopic apparatus of the present subject matter.

FIG. 10 depicts the connectivity of the movable rod and the movable and flexible element used in the articulation of the laparoscopic apparatus, along with a cross- sectional view and a perspective view of the movable operating element.

FIG. 11 depicts the movable element, in the form of a gripper, of the laparoscopic apparatus.

FIGS. 12 and 13 depict cross sectional views of the end effector assembly of the laparoscopic apparatus in an articulated position.

### DETAILED DESCRIPTION OF THE INVENTION

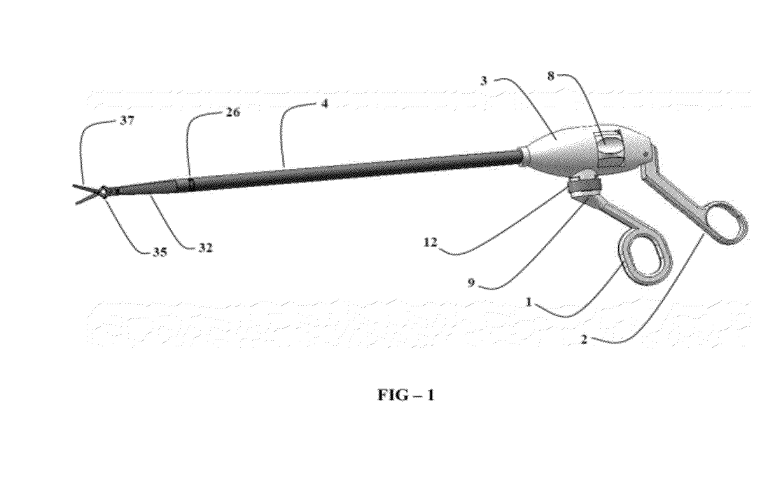

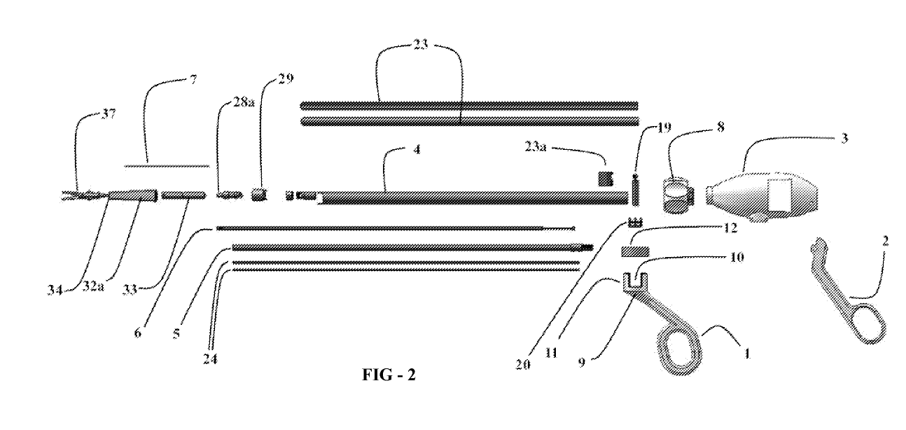

A laparoscopic apparatus, according to an aspect of the present subject matter, is described by referring to the accompanied figures. Initially, referring to the Figures as illustrated in FIGS. 1 through 2, the laparoscopic apparatus includes a fixed handle 1, a movable handle 2, and a pistol-shaped handle case 3 connected to a proximal end of an elongated barrel 4. The fixed and movable handles 1 and 2 are connected to the handle case 3, as shown in FIG. 1. Both the fixed and the movable handles 1 and 2 provide a grip to a user and enables the user to actuate other parts of the laparoscopic apparatus, for instance, during the course of laparoscopic surgery, as hereinafter described. The expression “proximal” as used above in the description indicates that portion of the laparoscopic apparatus which is normally close to a user while holding the laparoscopic apparatus. Similarly the expression “distal” indicates that portion of the laparoscopic apparatus which is farther away from the user.

Further, a rotatable conduit 5 with proximal and distal ends is disposed inside the elongated barrel 4 and extends axially from the movable handle 2 through the length of the elongated barrel 4. The proximal end of the rotatable conduit 5 is connected to a rotatable wheel 8. The rotatable conduit 5 is rotated by the user by means of the rotatable wheel 8.

A movable rod 6 with a ball-shaped proximal end is connected to the movable handle 2. Triggering movement of the movable handle 2 is translated into an axial movement of the movable rod 6. In one implementation, the movable rod 6 can also be arranged in conjunction with a tensioning device, like a spring, to provide a free play of the movable rod 6.

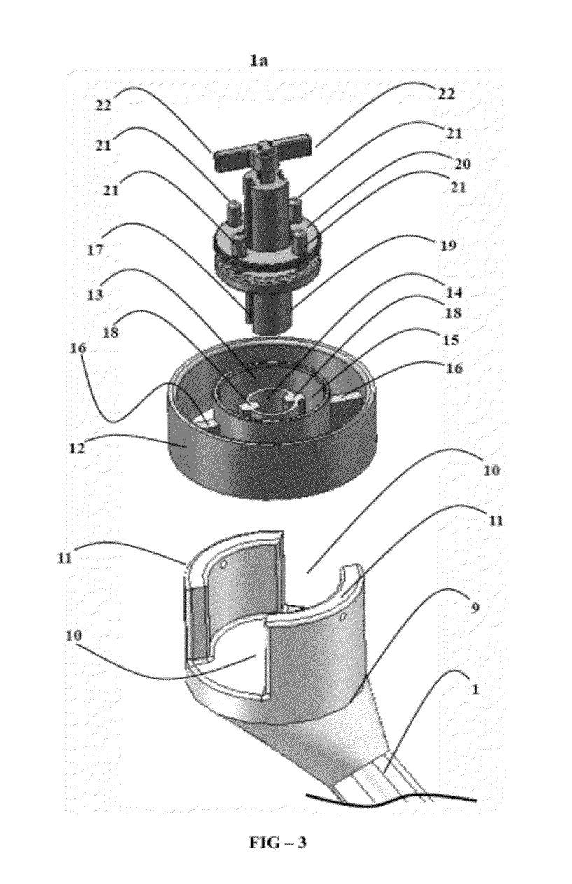

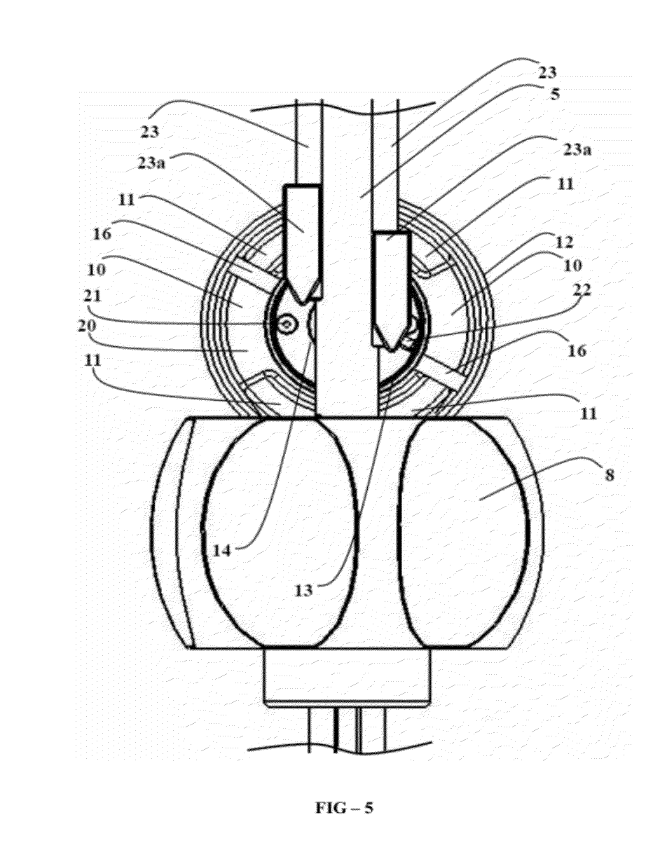

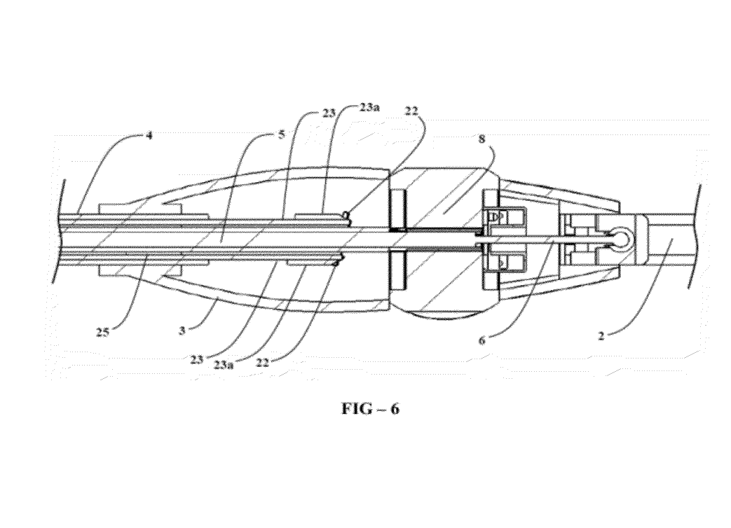

Now by referring to FIG. 3-6, the constructional arrangement of components to attain the wrist-like lateral movement of the laparoscopic apparatus is described. In one implementation, as shown in FIG. 3, an actuating assembly 1a that facilitates actuating motion of movable parts of the laparoscopic apparatus is described. The actuating assembly 1a includes the fixed handle 1 has a gripping portion and a holder 9 connected to the handle case 3. The holder 9 is provided with wall portions 11, which are designed to form peripheral slots 10. The wall portions 11 and the slots 10 are arranged in a manner to form an alternate arrangement of the wall portions 11 and the slots 10, on the periphery of the holder 9.

A holder ring 12 with a stopper element 16 is rotatably mounted on the outer periphery of the wall portions 11 of the holder 9 of the fixed handle 1. The holder ring 12 is disposed to move both in clockwise and counter clock-wise directions. The movement of the holder ring 12 is effected by the user. The stopper element 16 acts as a stopper to the movement of the holder ring 12. The stopper element 16 defines an extent of movement to the holder ring 12 in both clockwise and counter clock-wise directions. The holder ring 12, according to an embodiment, is a circular ring or a wheel. Any other compatible arrangements that can effect the desired circular movements can also be used as the holder ring 12.

A base ring 13 and an arm ring 14 with an intervening space 15 are disposed inside the holder ring 12. An inner surface of the arm ring 14 is covered with protrusions 18. A movable vertical support member 19, which is a stud-like structure, having longitudinal grooves 17 on its lateral surface and a base 20, is permeably fitted through the inner protrusions 18 of the arm ring 14. The connectivity of the movable vertical support member 19 with the holder ring 12 ensures the transmission of circular movement of the holder ring 12, which is effected by the user, to the movable vertical support member 19.

The fixed handle 1 is connected to the handle case 3 by means of projections 21 provided on the base 20 of the movable vertical support member 19.

Further, a pair of guide actuators 22, which are in the form of lateral projections, are mounted on the movable vertical support member 19. In one implementation, the projections have winged configuration; however, other suitable configurations can also be used for the guide actuators 22.

In another implementation, the holder ring 12, the base ring 13, the arm ring 14, the movable vertical support member 19, and the guide actuators 22 can be made from any metal or alloy or any wear resistant polymers.

A space 25 is arranged inside the elongated barrel 4 and rotatable conduit 5. The space 25 extends through the length of the elongated barrel 4.

The laparoscopic apparatus further includes at least one pair of immovable guide member supporters 24, which is axially disposed inside the space 25 between the elongated barrel 4 and the rotatable conduit 5. In one embodiment, the immovable guide member supporters 24 are disposed on top and bottom inner surfaces of the elongated barrel 4. The guide member supporters 24 are elongated slides or strips extending from the rotatable wheel 8 to the distal end of the elongated barrel 4.

Movable guide members 23, in the form of elongated slides or strips having proximal and distal ends, are movably and axially disposed in the space 25 between the elongated barrel 4 and the rotatable conduit 5. The proximal ends 23(a) of the movable guide members 23 are abutted to the guide actuators 22. Accordingly, whenever the guide actuators 22 are actuated, the corresponding motion is transmitted to the movable guide members 23 so that the movable guide members 23 move axially through the elongated barrel 4.

Therefore, the movable guide members 23 are disposed to possess a relative axial movement, inside the space 25, of the elongated barrel 4 and the rotatable conduit 5, on being actuated by the rotary motion of the movable guide members 23.

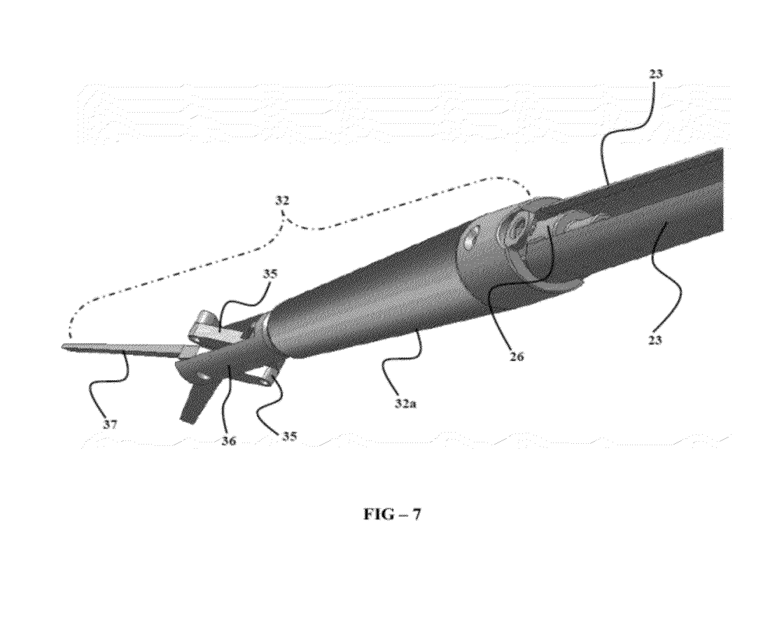

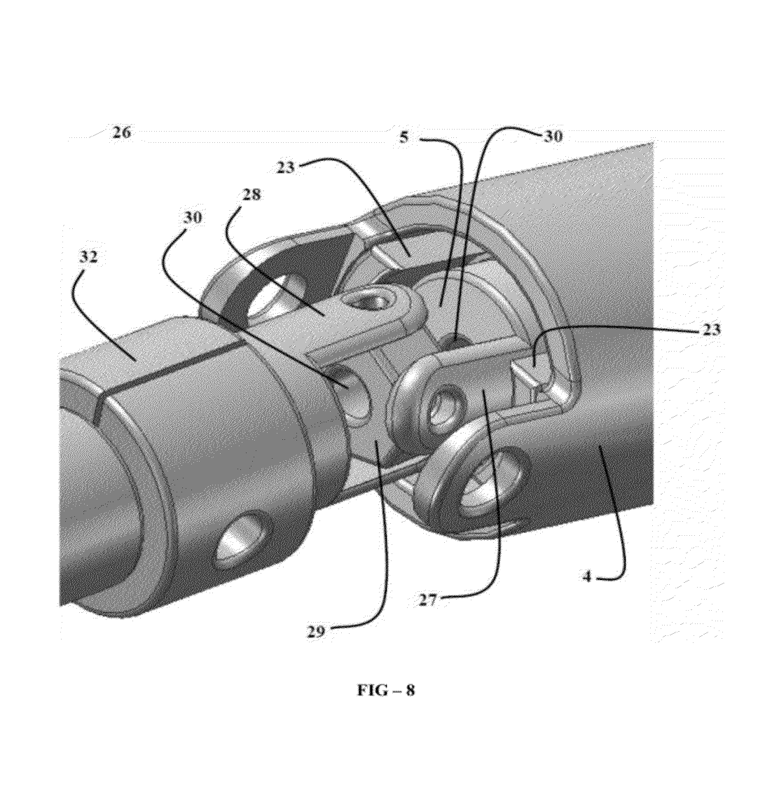

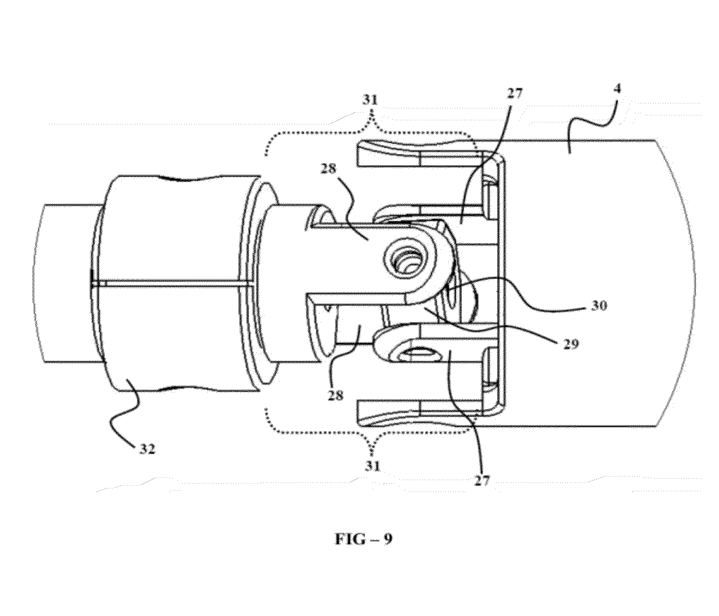

Now by referring to FIGS. 7-11, the constructional features of a universal joint 26 and its integration with the other parts of the laparoscopic apparatus are described.

The universal joint 26 includes a pair of rotatable universal joint arms 27 connected to the rotatable conduit 5 on one side and to a movable universal joint arm link 29 having a passage 30, on the other side. Consequently, the universal joint arms 27 and the movable universal joint arm link 29 are disposed to rotate in conjunction with the rotation of the rotatable conduit 5 upon the rotation of the holder ring 12. Further, a pair of movable universal joint arms 28 is connected to the movable universal joint arm link 29. The connectivity of the rotatable universal joint arms 27, the movable universal joint arm link 29, and the movable universal joint arms 28 form the universal joint 26 to provide a flexible and movable connecting link between the elongated barrel 4 having the movable guide members 23, the rotatable conduit 5, and the movable rod 6 on one side and a movable end-effector 32 on the other side, as hereinafter described.

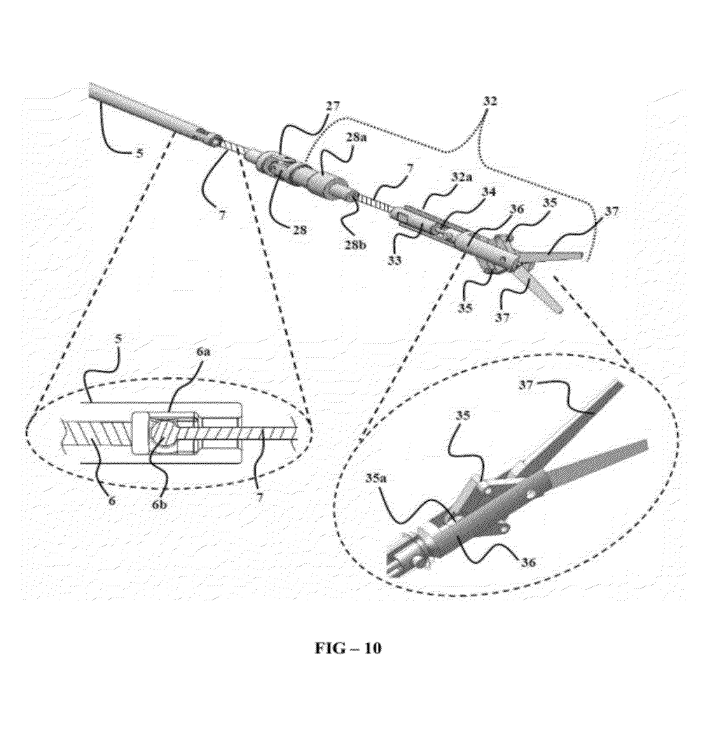

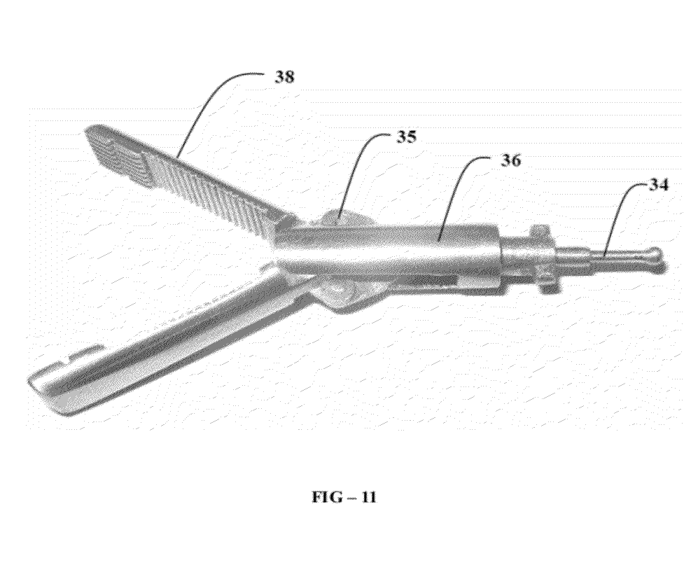

The movable end-effector 32 includes an end-effector housing 32a. The end-effector housing 32a includes a cylinder 28a having a passage 28b, which is connected to the movable universal joint arms 28. A movable piston 33 is connected to the cylinder 28a in a spatial arrangement by means of a flexible and movable element 7, as hereinafter described. A movable piston rod 34, which is also enclosed in the end-effector housing 32a, is connected to the movable piston 33. Further, a movable arm supporting member 36 is connected to the end-effector housing 32a. Also, a movable piston rod 34 is connected to the movable piston 33 to receive the axial motion from the movable piston 33. The movable piston rod 34 is arranged in a manner to extend from the end-effector housing 32a to the movable arm supporting member 36. Movable arms 35 are movably connected at a converging point 35a of the movable arms 35. The movable arms 35 are pivoted to the movable arm supporting member 36, which is connected to the end-effector housing 32a. The converging point 35a acts like a pivot for the movable arms 35. Further, a pair of movable operating element 37 is linked to the movable arms 35. The movable operating element 37 is held by the movable arm supporting member 36 with a riveting arrangement as shown in FIG. 10. In one implementation of the present embodiment, the movable operating element 37 is implemented as a pair of scissors. However, in another embodiment, depicted in FIG. 11, the movable operating element 37 may be implemented as a gripper 38.

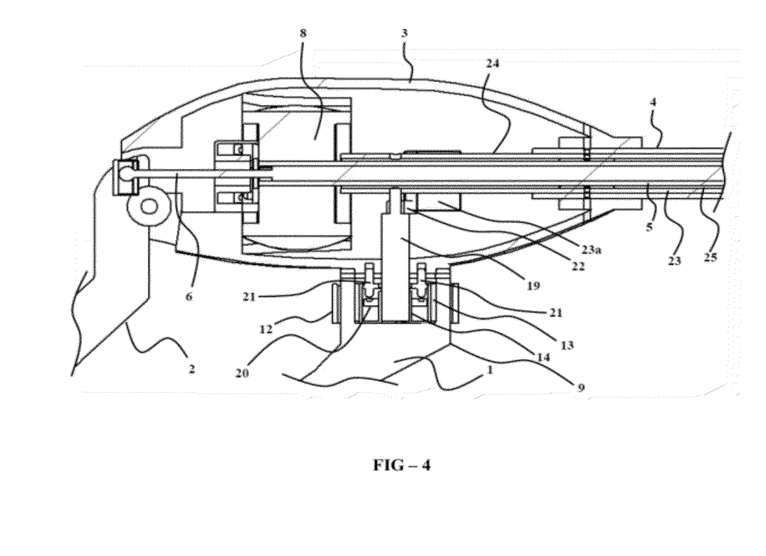

The movable and flexible element 7, which can be a flexible wire or a cable, is coupled to the distal end of the movable rod 6 on one side and to the movable piston 33 on the other side, as shown in FIG. 10. The movable and flexible element 7 has a ball-shaped end 6b, which is press-fitted into a coupler 6a having a corresponding cavity. An identical coupling arrangement is also implemented on the other end of the movable and flexible element 7 when it is connected to the movable piston 33. Further, the proximal end of the movable rod 6 is connected to the movable handle 2 as shown in FIG. 4. The movable and flexible element 7 originating from the distal end of the movable rod 6 is permitted to pass through the universal joint arm link passage 30 of the universal joint 26. The movable and flexible element 7, which is connected to the movable piston 33, is further connected to movable piston rod 34, which in turn is connected to the movable arms 35 and the corresponding movable operating element 37.

Accordingly, by referring again to FIG. 10, when the user operates the laparoscopic apparatus for a surgery, the movable handle 2 is triggered and the resultant axial motion is transmitted through the movable rod 6. The axial movement of the movable rod 6 is further transmitted to the movable and flexible element 7, which in turn activates the movable piston 33 by pulling the movable piston 33 towards the proximal end of laparoscopic apparatus. Consequently, the movable piston rod 34, which is connected to the movable piston 33, pulls the movable arms 35 so that movable arms 35 move closer to each other, resulting in closing of the movable operating element 37. Thereafter, whenever the movable piston rod 34 is released or moved towards the distal end, due to release of the movable handle 2, the movable arms 35 respond by moving away from each other, thus resulting in opening of the movable operating element 37. The co-operative movements of opening and closing of the movable arms 35 facilitate operations of the movable operating element 37, in the form of either gripping or cutting the desired tissue/organ during the course of the laparoscopic surgery.

An end-effector actuating space 31 is arranged between the elongated barrel 4 and the movable end-effector 32. The universal joint 26 is disposed in the end-effector actuating space 31. Further, the end-effector actuating space 31 allows the movable universal joint arms 28 to move axially and actuate the movable end-effector 32 by pushing the movable end-effector 32 to the desired lateral wrist-like movement.

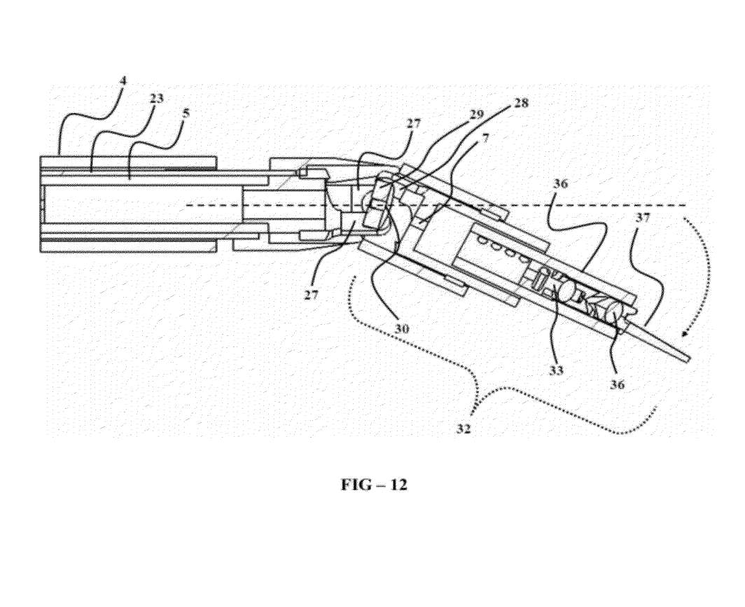

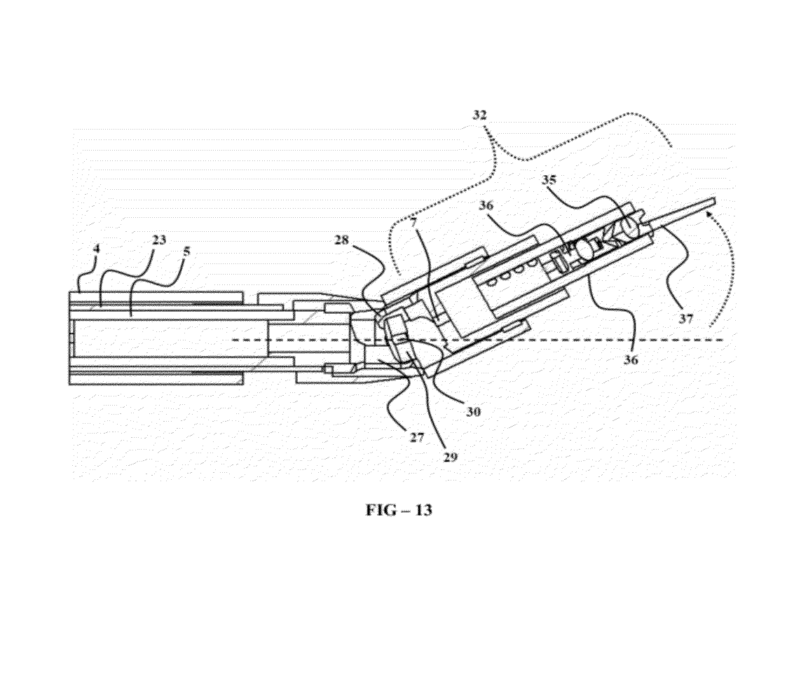

The lateral wrist-like movement of the movable end-effector 32 is now described by referring FIGS. 12 and 13. In one implementation, the articulation of the holder ring 12 actuates the movable guide members 23. The clock-wise and the counter clock-wise rotations of the holder ring 12 actuates the axial movement of the corresponding movable guide members 23. The axial movement of the movable guide members 23 extends into the end-effector actuating space 31 and pushes the movable end-effector 32, in a pre-determined direction, corresponding to the clock-wise and the counter clock-wise rotary movement of the holder ring 12, thereby causing the lateral wrist-like movement of the movable end-effector 32.

The laparoscopic apparatus of the present invention possesses a workable range of angle of articulation of the movable end-effector 32 along with a rotation of 360° about the axis of the elongated barrel 4.

The laparoscopic apparatus, during the course of a laparoscopic surgery, enables the user not only to hold and cut the selected subject but also simultaneously enables the lateral wrist-like movement along with rotary movement of the end-effector 32.

The laparoscopic apparatus has a decoupling arrangement in the form of separation of various functions of the laparoscopic apparatus viz., lateral wrist like motion of the movable end-effector 32, cutting and gripping arrangement of the movable operating element 37, and rotary motion of the movable operating element 37, which are effected by the combined operation of movable end-effector 32 and the universal joint 26 and the handle assemblies as shown in corresponding representative FIGS. 7-13.

Therefore, the laparoscopic apparatus of the present invention provides an improved articulation with an extra degree of freedom, while retaining its existing surgical functions. The end effector of the laparoscopic apparatus is provided with a wrist-like lateral movement to provide desired extra degree of freedom.

The laparoscopic apparatus of the present invention is provided with independent actions for lateral wrist-like motion, cutting and gripping arrangement and rotary motion.

The movable end-effector can be easily detachable from the body of the laparoscopic apparatus. The laparoscopic apparatus has an extra degree of freedom that enhances the dexterity of a user in a minimally invasive surgery.

While there have been described what are presently believed to be the preferred embodiments of the invention, those skilled in the art will realize that various changes and modifications may be made to the invention without departing from the spirit of the invention, and it is intended to claim all such changes and modifications as fall within the scope of the invention.

We claim:

1. A laparoscopic device for performing surgical functions on a selected object, comprising;
a fixed handle, a movable handle, and an elongated barrel, connected to a handle case,
a rotatable conduit disposed inside said elongated barrel and connected to a rotatable wheel of said handle case,
a movable rod disposed inside said rotatable conduit and connected to said movable handle,
characterized in that
a movable end-effector functionally
(a) connected to said movable handle,
(b) connected to said rotatable wheel, and
(c) connected to an actuating assembly of said fixed handle, and
a universal joint functionally
(a) connected to said movable end-effector on one side
(b) connected to said movable handle, to said rotatable wheel, and to said actuating assembly, on the other side,
wherein said universal joint disposed to permit independent and simultaneous actuation of said movable handle, said rotatable wheel and said actuating assembly.

2. The laparoscopic device as claimed in claim 1, wherein said movable end-effector comprising;
a cylinder with a passage,
a movable piston disposed in an end-effector housing connected to said cylinder through a movable and flexible element, wherein said movable and flexible element functionally connected to said movable rod on the other side,
a movable piston rod connected to said movable piston on one side and movable arms on the other side, wherein said movable arms pivoted to a movable arm supporting member, and
a movable operating element operably connected to said movable arms.

3. The laparoscopic device as claimed in claim 2, wherein said movable and flexible element is a wire or cable.

4. The laparoscopic device as claimed in claim 2, wherein said movable operating element is scissors or a gripper.

5. The laparoscopic device as claimed in claim 1, wherein said actuating assembly comprising;
a holder of said fixed handle with slots and wall portions, a movable holder ring mounted on said wall portions of said holder, a movable vertical support member with guide actuators connected to said holder ring, movable guide members disposed in said elongated barrel, wherein said guide actuators functionally connected to said movable end-effector through said movable guide members.

6. The laparoscopic device as claimed in claim 5, wherein said movable holder ring comprising;
a base ring, an arm ring with intervening space, and a stopper element rotatably mounted on said wall portions of said holder and disposed between said holder ring and said base ring.

7. The laparoscopic device as claimed in claim 1, wherein said universal joint comprising;
a pair of rotatable universal joint arms connected to said rotatable conduit, a pair of movable universal joint arms connected to said movable end-effector, a movable universal joint arm link with a passage disposed to connect said rotatable universal joint arms and said movable universal joint arms, and wherein said universal joint disposed in an end-effector actuating space.
